(19) United States
(12) Patent Application Publication
(10) Pub. No.: US 2020/0245851 A1
(43) Pub. Date: Aug. 6, 2020

(54) CASE FOR THE STERILIZATION AND CONSERVATION OF THERMOLABILE ENDOSCOPES

(71) Applicant: MEDIQUA SRL, Albano Laziale (RM) (IT)

(72) Inventors: Tiziano FRACASSI, CAPANNORI (LU) (IT); Fabrizio RAFFAELLI, CASCINA ALTA/CASCINA TERME LARI (PI) (IT)

(21) Appl. No.: 16/648,862

(22) PCT Filed: Sep. 20, 2018
(86) PCT No.: PCT/IT2018/050172

§ 371 Date: Mar. 19, 2020

(30) Foreign Application Priority Data
Sep. 28, 2017 (IT) 102017000108592

(51) Int. Cl. A61B 1/00; A61B 1/12; A61L 2/24; A61L 2/18

(52) U.S. Cl. CPC A61B 1/00142; A61B 1/125; A61L 2/24; A61L 2202/24; A61B 1/00121; A61B 1/00137; A61L 2/18

(57) ABSTRACT

Disclosed is a case for the sterilization and conservation of thermolabile endoscopes for use, in cooperation with an automatic machine, for washing and sterilizing one of the endoscopes including multiple channels. The case includes: first and second shells; a reversible fastener between the first and second shells; connectors for the channels of the endoscope, allowing injection of process liquids and/or gas into the channels; an inlet connector and an outlet connector, allowing injection and discharge of process liquids and/or gas into and out of the case; a handle and supporting feet. The first and second shells reproduce in the plan view a shape with a substantially circular overlapping edge. The surface of the first and second shells includes a convex portion near the edge and a concave portion in the central zone, where the portions are connected to one another with a curved profile, free of corners.

### FIELD OF APPLICATION

The present invention relates to the biomedical sector and, more specifically, to the processes of disinfection, and preferably of sterilization, of medical instruments. In detail, the invention relates to a case for the sterilization and conservation of thermolabile endoscopes.

### STATE OF THE ART

Every endoscopic examination must be free of the risk of transmission of infections, i.e. every instrument or accessory used must be free of any infective load that could contaminate the patient. Infective risks associated with an endoscopic procedure are in fact correlated to the re-use of the endoscopes used in daily practice, which pose the risk of exposure to bodily fluids (blood, pus, etc.) and to transmissible pathogenic organisms.

Every medical instrument must therefore be re-processed after each use.

In particular, the re-processing of endoscopes can be defined as the set of all procedures involved in the treatment of the instruments, the objective being to ensure safe performance for the patient and for the operator, and substantially comprises the operations of:
pre-washing;
manual cleaning (cleansing);
rinsing;
high-level disinfection and sterilization;
rinsing;
drying;
storage;
transport: from the (external or internal) sterilization unit to “the patient's side” and vice-versa.

Thorough manual cleaning, with the mechanical removal of the greatest possible quantity of organic material, is the essential pre-requisite for a correct disinfection and sterilization process.

Above all, it is necessary to distinguish what the (high-level) disinfection and preferable sterilization consist of.

A high-level disinfection guarantees the neutralization of all bacteria and viruses, but only of a limited number of spores (bacterial load abatement with a function of 10⁻⁵); sterilization on the other hand is a process which completely eliminates all forms of microbial life and all spores (bacterial load abatement with a function of 10⁻⁶).

Current standards only call for high-level disinfection of thermolabile endoscopes and their subsequent conservation in an aseptic environment; however, the problems associated with the possible transmission of pathogens make it desirable to further raise the quality of required purification, to achieve the complete and verifiable sterilization of all equipment, including thermolabile endoscopes.

Cleaning of endoscopes initially used to be a manual procedure, which could not be standardized, with the risk of incomplete elimination of all the bacterial loads and resultant infectious and/or chemical risks for the patient and for the operator.

The introduction into clinical practice of modern automatic equipment, i.e. endoscope-washer machines, has automated procedures for high-level disinfection and/or sterilization of endoscopes, making it possible to achieve standardization of the various stages of re-processing, thereby reducing the risks for health operators and raising the safety profile of the sterilization process.

These machines operate by making the external surface and the internal channels of the endoscope come into contact with washing and sterilization liquids.

The internal and external surfaces of the endoscope, the valves and accessories then need to be thoroughly dried in order to avoid the re-growth of any micro-organisms present in the residual water.

Also known are cabinets fitted with filters that are able to maintain an internal environment (temperature, humidity) capable of preventing the proliferation of pathogenic micro-organisms during prolonged storage periods.

These processes have proved to be effective with non-thermolabile endoscopes, and to be problematic with thermolabile endoscopes, especially when it comes to the stages of maintenance/storage/transport of the thermolabile endoscope from the environment in which the disinfection and/or sterilization process was carried out, to the “patient's side”.

Patent application No. RM2003A000570 describes a system for the washing, sterilization and conservation of endoscopes.

In detail, this system involves the endoscopes being placed, during the washing and sterilization stages carried out in endoscope-washer machines, inside special hermetically sealed cases, and these cases are then placed in the conservation cabinets, adapted to guarantee a sterile environment over time.

These cases are made of two hinged shells, which in the plan view have a substantially rectangular shape; they are made of a plastic material (specifically polycarbonate); they comprise spring-type point closing means.

Each case is provided with connectors for the introduction of washing or sterilization liquids and/or flushing or drying gases, into the case compartment and into the individual channels of the endoscopes.

Each case is also provided with an outlet connector for draining the liquids at cycle end, leaving the interior compartment in a sterile condition.

The shape and the material of which these cases are made makes them fragile, stiff and non-deformable, and they cannot therefore withstand internal pressure variations, due to the introduction and extraction of liquids and gases for washing, sterilization and drying, that substantially exceed the atmospheric pressure in the pressure cycles.

These cases also do not guarantee impermeability against external agents: if it is not possible to maintain a high internal pressure, greater than atmospheric pressure, it could be easy for contaminated air to find its way inside the case.

Furthermore, known types of containment cases do not ensure the complete removal of water residues and/or water vapor during the drying stage, since that can only be guaranteed either by means of heat treatments (heating to create evaporation) that cannot be used on thermolabile endoscopes, or with vacuum cycles approaching pressures of less than 100 millibars.

### Presentation of the Invention

The object of the invention is to overcome these problems. In particular, the invention proposes to provide a case for the sterilization and conservation of thermolabile endoscopes usable with endoscope-washer machines to guarantee sterilization and facilitate the re-processing procedures to which thermolabile endoscopes are subjected on a daily basis, and to maintain sterile conditions on the inside over time, thereby avoiding the use of sterile cabinets.

The objects are achieved with a case for the sterilization and conservation of thermolabile endoscopes adapted to be used, in cooperation with an automatic machine, in a process of washing and sterilizing one of said endoscopes comprising a plurality of channels, where said case comprises:
a first shell;
a second shell;
reversible fastening means between said first and said second shell;
a plurality of connectors for the channels of said endoscope, adapted to allow the injection of process liquids and/or gas directly into said channels;
an inlet connector and an outlet connector, adapted to allow the injection and discharge of process liquids and/or gas into and out of said case;
a handle;
supporting feet,

characterized in that said first and said second shell reproduce in the plan view a shape with a substantially circular overlapping edge, and the surface of said first and said second shell comprises a convex portion placed in the proximity of said edge and a concave portion in the central zone, o where said portions are connected to one another with a curved profile, free of corners.

Advantageously, said first and said second shell are made from elastically deformable material.

In a possible variant of the invention, said first and said second shell have different depths.

Advantageously, only one of said first or said second shell comprises said plurality of connectors and said inlet connectors and outlet connectors.

According to one aspect of the invention, said elastically deformable material is a composite material.

In particular, said composite material comprises at least one first layer made of carbon fiber in a synthetic resin matrix.

In a possible variant of an embodiment, said composite material comprises at least one second layer made of carbon fiber in a synthetic resin matrix, and an intermediate layer of lightening material.

Furthermore, said composite material also comprises fibers chosen from fibers of Kevlar, glass, or metallic wires.

According to another aspect of the invention, said first and said second shell have an average thickness of between 1 and 4 mm, preferably between 2 and 3 mm.

In a preferred variant of embodiment, said plurality of connectors are grouped together in a single-piece structure so as to be univocally associable with a corresponding complementary structure belonging to said automatic machine.

According to a further aspect of the invention, one of said first or said second shell comprises along its edge a channel for containing a seal, where said seal is adapted to cooperate with a corresponding lip on the edge of the other shell to keep the interior of said case insulated from the outside.

Furthermore, said reversible fastening means comprise a strip of inextensible material provided with tightening means, adapted to tighten the edges of said first and said second shell to keep them stably joined to one another.

Advantageously, said strip comprises said handle and said supporting feet.

In a preferred variant, said strip comprises means for form coupling with said machine.

According to another aspect of the invention, said case for the sterilization and conservation of thermolabile endoscopes comprises a system for the identification and/or automatic storage of information relating to said case and/or said endoscopes.

The advantages of the invention are multiple.

The case described by the invention has the ability to withstand vacuum cycles (suction of air, liquids, residues of humidity from the inside towards the outside) until it reaches an internal pressure or around one thousandth of the external pressure, in a repeated and measurable manner.

The case also has the ability to withstand pressurization cycles (the introduction of purified/sterile air from the outside to the inside) with the creation of an internal environment with a pressure value equivalent to around one and a half times the external (atmospheric) pressure, also in a repeated and measurable manner.

The substantially circular and symmetrical geometry of the two shells that make up the case, with convexity along the edge and concavity at the center, makes it possible to optimize the deformability of the case, making this deformability controlled, measurable and repeatable.

The use of a suitable elastically deformable material also enables the case to withstand large positive and negative variations in internal pressure.

Indeed, during vacuum cycles the case deflects in a geometrically uniform manner both at the center (the concave part, to a greater degree) and at the convex parts near the outer edge (to a lesser degree), while during pressurization cycles it expands (inflates) in an equal and opposite manner with respect to the vacuum cycles, even if with less movement (around half the millimetric movement between vacuum and pressure).

The case described by the invention permits the use of processes involving a vacuum and processes where pressures greater than atmospheric pressure are applied, creating an internal environment, containing the endoscope, that at the end of the process does not come back into contact with the external, potentially contaminating environment until the endoscope is used.

The case can therefore be used for the storage of endoscopes even in the presence of a non-sterile external environment, and for the transport of said endoscopes from the washing-sterilization environments to the storage environments, and as far as the environments used for medical examinations, i.e. to “the patient's side”.

### BRIEF DESCRIPTION OF THE DRAWINGS

Further features and advantages of the invention will be more evident from the more detailed description set forth below, with the aid of the drawings, which show a preferred implementation thereof, illustrated by way of non-limiting example, wherein:

FIGS. 1-4 show a plan view from above and from below and two side views respectively, of a case for the sterilization and conservation of thermolabile endoscopes according to the invention;

FIG. 5 shows a cross-section along a partially exploded vertical plane of the case shown in FIGS. 1-4.

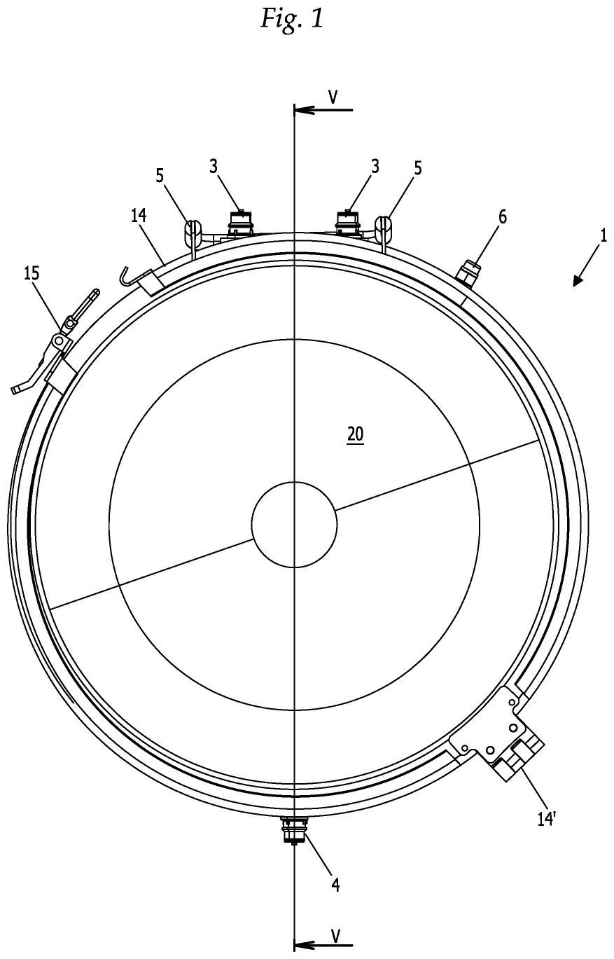

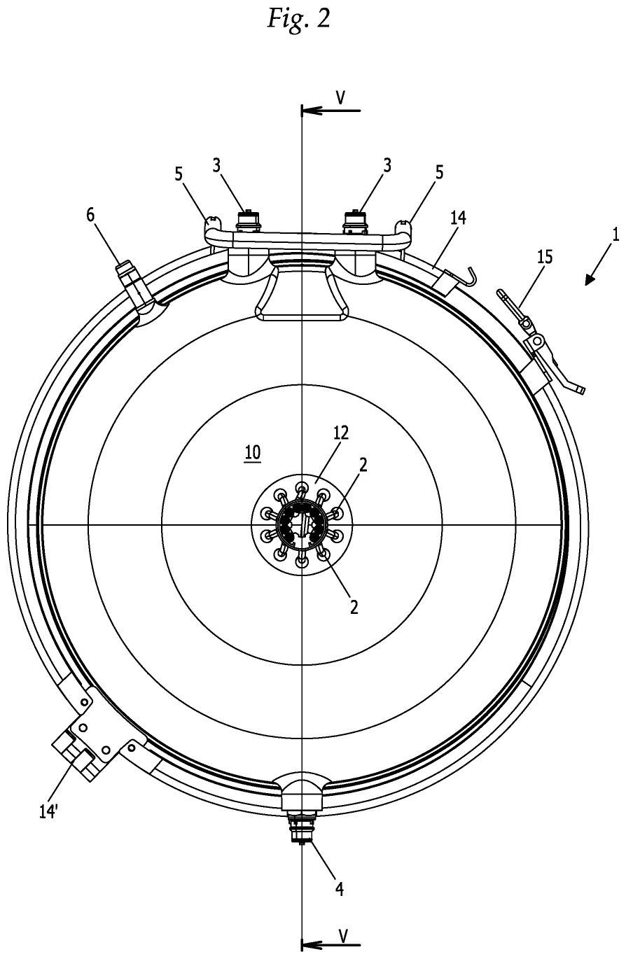

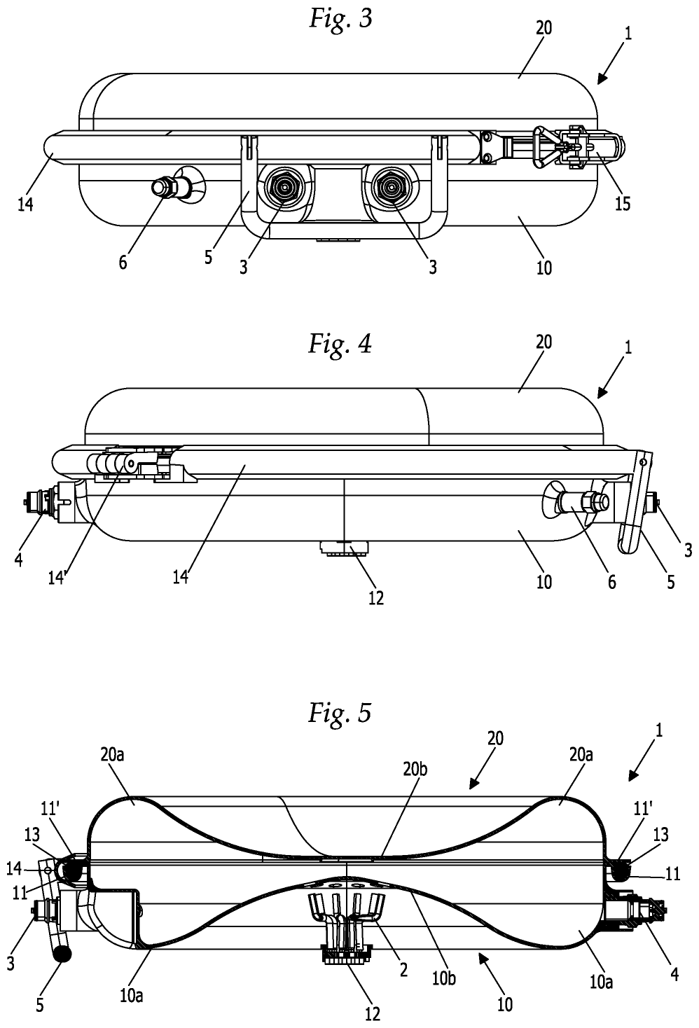

### DETAILED DESCRIPTION OF A PREFERRED EMBODIMENT OF THE INVENTION

With reference to the Figures, the invention relates to a case 1 for the sterilization and conservation of thermolabile endoscopes adapted to be used, in cooperation with an automatic machine of the “endoscope-washer” type, in a process of washing and sterilization.

Said case 1 comprises a first shell 10 and a second shell 20 which serve the interchangeable functions of base and cover, adapted to create a closed containment compartment, intended to accommodate within it any type of thermolabile endoscope.

Said case 1 also comprises reversible fastening means between said first 10 and said second 20 shell.

Said first 10 and said second 20 shell reproduce in the plan view a substantially circular shape. The shape of a perfect circle is preferable but not limiting, for example even an elliptical shape would enable the same advantages to be achieved.

The edges of the two circular shapes must necessarily correspond, since the two shells 10, 20 must be joined together in order to perform their intended purpose, i.e. acting as a container.

Said first shell 10 comprises along its edge a channel 11 for containing a seal 13.

Said seal 13 is adapted to cooperate with a corresponding lip 11′ on the edge of said second shell 20 to keep the internal environment of said case 1, where the endoscope is stored, insulated from the outside.

The internal environment and the external environment will be isolated from one another during washing and rinsing cycles (introducing liquids into the case), and during drying, vacuum and pressurization cycles.

In the variant shown, said first 10 and said second 20 shell have different depths.

Said first 10 and said second 20 shell are provided with a convex portion 10a, 20a along their outer edge, and a concave portion 10b, 20b at the center; the point of maximum concavity is generally the point furthest from the outer edge.

Said first 10 and said second 20 shell are made of elastically deformable composite material.

Said composite material comprises at least one layer made of carbon fibers in a synthetic resin matrix, or carbon fibers mixed and woven with fibers made of another material (e.g. Kevlar, glass fiber).

Depending on the mechanical properties required, the fibers can be woven in a warp and weft manner, in a single direction, or else with a 45° weave.

A preferred variant of the case has the shells made from a material comprising at least ⅔ carbon fiber.

Carbon fibers can have an elastic modulus of between 240 and 670 GigaPascal.

Excellent results have been obtained by using pitch fibers K13312 Dialed Mitsubishi, K63712 Dialed Mitsubishi, K63916 Dialed Mitsubishi, K13916 Dialed Mitsubishi, and fibers of PAN (polyacrylonitrile) 34-700 Grafil Mitsubishi, STS 40 F13 TohoTenax Teijin to weave the raw materials.

Preferably, but not exclusive, said resin is an epoxy resin.

In possible variants of embodiment not shown, said first and said second shell are made with a sandwich structure, comprising two outer layers made of a composite material in carbon fiber and synthetic resin, joined to one another with an intermediate layer of lightening material.

By way of example, said filling material may be chosen from Termanto polyurethane foam of the Lantor SoricR type or Lantor CorematR, PMI (polymethalcrylimide) RohacellR by Evonik, PET (polyethylene terephtalate) by 3D CORE GmbH, FlexcoreR aluminum honeycomb by Imatec, and aramid paper impregnated with NomexR phenolic resin by Imatec.

The shells 10, 20 must have an average thickness of between 1 and 4 mm, preferably between 1.5 and 3.5 mm and even more preferably between 2 and 3 mm.

More generally, the shells 10, 20, and therefore the case 1, can be constructed using pre-impregnated fabrics of fibers and resin, to be modeled on molds (metallic or carbon or silicon), using the “molding” technique, with polymerization of the composite material in an autoclave or an oven.

Alternatively, the shells 10, 20 may be constructed using dry fabrics of carbon fiber or carbon plus other materials, such as Kevlar, glass fiber, or metallic wires, and can be modeled on press molds or injected with resin during press molding while heating the mold (RTM—Resin Transfer Molding technique).

Said case comprises:
a plurality of connectors 2 with which the individual channels of said endoscope are directly associable, adapted to allow the injection of process liquids and/or gas directly into said channels;
a pair of inlet connectors 3 and an outlet connector 4, adapted to allow the injection and discharge of process liquids and/or gas into and out of said case 1, i.e. into the confinement compartment defined by the two shells 10, 20;
a bleed valve 6 acting as a safety valve in the event of excess pressure;
a handle 5;
supporting feet (not illustrated).

Advantageously, in the variant shown, said plurality of connectors 2 and said inlet connectors 3 and outlet connectors 4 are all placed on said first shell 10. In this way only one shell è “technologized”, substantially the shell acting as the “base” during when inserting and/or taking the endoscope. Production of the shells is thereby facilitate and optimized, since all the seconds shells, those acting as a “cover” for the case, can be produced in series, more quickly and with appropriate depths. It should be stressed that the concept of “base” and “cover” is used only to make the shells recognizable, since during the sterilization process on a machine the case 1 remains in a vertical position with the inlet connectors 3 and outlet connectors 4 for process liquids and gases placed logically and respectively at the top and bottom, while during storage of the case 1, it does not make any difference which position it is resting in.

Even more advantageously, said plurality of connectors 2 are grouped together in a single-piece structure 12, shown in an exploded view in FIG. 5, so as to be univocally associable with a corresponding complementary structure belonging to said machine, from which the process liquids and/or gases will be fed or aspirated.

Said reversible fastening means comprise a strip 14 of inextensible material, for example a metallic strip, provided with tightening means 15 that can be length adjusted using known screw-type systems, adapted to tighten the edges of said first 10 and said second 20 shell to keep them stably joined to one another. Said strip 14 is divided into two portions connected to one another by a hinge 14′.

Said strip 14 is a sort of belt, which fits around the edges of the two shells 10, 20, when they are joined together.

In the variant shown, said handle 5 (and possibly said supporting feet) are provided directly on said strip 14. In this case, too, production of the shells 10, 20 is simplified, since all additional components of the case 1 do not interfere with their shape.

In a possible embodiment, said strip 14 could comprise uniquely shape means of joining with said endoscope-washer machine, in order to avoid possible orientation errors by the operator when placing the case 1 in the machine.

Advantageously, in a possible variant of the invention (not illustrated), said case 1 could comprise a system for the identification and/or automatic storage of information relating to the case or the endoscopes it contains, in order to enable recognizability and identification and tracking of the processes undergone. This system could be of an RFID (radio frequency identification) type.

By way of non-limiting example, several measurements are given for the components that make up the case 1 according to the invention.

Considering the perfectly circular shape to be preferred, but not absolutely essential, the external diameter of the two shells 10, 20 can be defined as the line segment passing through the center and joining two points on the outer edge. In the case of a non-perfectly circular shape, the external diameter of the two shells is defined as the line segment passing through the center(s) and joining the two points on the outer edge that are farthest apart from one another.

The suggested measurement for this external diameter is between 600 and 400 mm, preferably between 550 and 450 mm, and even more preferably between 540 and 520 mm.

The measurement for the maximum overall height of the case is defined as being between 100 and 200 mm, preferably between 120 and 180 mm, and even more preferably between 140 and 160 mm. The minimum height, measured substantially as the distance between the two central points of maximum concavity, is defined as being between 10 and 50 mm, preferably between 15 and 40 mm, and even more preferably between 25 and 30 mm.

As mentioned above, the elasticity of the material used and the resistance of the two shells to positive pressure and negative pressure, enables the case to withstand heavy-duty processes of washing, sterilization and drying of the endoscopes.

By way of example, a re-processing cycle is described for thermolabile endoscopes, consisting of a series of alternating and repeated work cycles, made possible by the characteristics of the case 1 described.

This cycle comprises the steps of:
external and internal washing (the term ‘internal’ refers to the internal channels of the endoscope, while the term ‘external’ refers to all visible surfaces of the endoscope);
Drying
disinfection (bacterial load abatement with a function of 10⁻⁵);
sterilization (bacterial load abatement with a function of 10⁻⁶);
vacuum cycles (bringing the pressure inside the case to less than 5 millibars, and preferably but not necessarily down to 1 millibar);
pressurization cycles (increasing the pressure inside the case with respect to the external atmospheric pressure, to a value of between 1.3 and 1.7 times the external atmospheric pressure);
maintaining the increased internal pressure;
storage;
transport.

1. A case (1) for the sterilization and conservation of thermolabile endoscopes adapted to be used, in cooperation with an automatic machine, in a process of washing and sterilizing one of said endoscopes comprising a plurality of channels, where said case (1) comprises:
a first shell (10);
a second shell (20);
reversible fastening means between said first (10) and said second (20) shell;
a plurality of connectors (2) for the channels of said endoscope, adapted to allow the injection of process liquids and/or gas directly into said channels;
an inlet connector (3) and an outlet connector (4), adapted to allow the injection and discharge of process liquids and/or gas into and out of said case (1);
a handle (5);
supporting feet,
wherein said first (10) and said second (20) shell reproduce in the plan view a shape with a substantially circular overlapping edge, and the surface of said first (10) and said second (20) shell comprises a convex portion (10a, 20a) placed in the proximity of said edge and a concave portion (10b, 20b) in the central zone, where said portions (10a, 10b, 20a, 20b) are connected to one another with a curved profile, free of corners.

2. The case (1) for the sterilization and conservation of thermolabile endoscopes according to claim 1, wherein said first (10) and said second (20) shell are made from elastically deformable material.

3. The case (1) for the sterilization and conservation of thermolabile endoscopes according to claim 1, wherein said first (10) and said second (20) shell have different depths.

4. The case (1) for the sterilization and conservation of thermolabile endoscopes according to claim 1, wherein one of said first (10) or said second (20) shell comprises said plurality of connectors (2) and said inlet connectors (3) and outlet connectors (4).

5. The case (1) for the sterilization and conservation of thermolabile endoscopes according to claim 2, wherein said elastically deformable material is a composite material.

6. The case (1) for the sterilization and conservation of thermolabile endoscopes according to claim 5, wherein said composite material comprises at least one first layer made of carbon fiber in a synthetic resin matrix.

7. The case (1) for the sterilization and conservation of thermolabile endoscopes according to claim 6, wherein said composite material comprises at least one second layer made of carbon fiber in a synthetic resin matrix, and an intermediate layer of lightening material.

8. The case (1) for the sterilization and conservation of thermolabile endoscopes according to claim 6, wherein said composite material also comprises fibers chosen from fibers of Kevlar, glass, or metallic wires.

9. The case (1) for the sterilization and conservation of thermolabile endoscopes according to claim 1, wherein said first (10) and said second (20) shell have an average thickness of between 1 and 4 mm.

10. The case (1) for the sterilization and conservation of thermolabile endoscopes according to claim 9, wherein said first (10) and said second (20) shell have an average thickness of between 2 and 3 mm.

11. The case (1) for the sterilization and conservation of thermolabile endoscopes according to claim 1, wherein said plurality of connectors (2) are grouped together in a single-piece structure (12) so as to be univocally associable with a corresponding complementary structure belonging to said machine.

12. The case (1) for the sterilization and conservation of thermolabile endoscopes according to claim 1, wherein one of said first (10) or said second (20) shell comprises along its edge a channel (11) for containing a seal (13), where said seal (13) is adapted to cooperate with a corresponding lip (11′) on the edge of the other shell (20, 10) to keep the interior of said case (1) insulated from the outside.

13. The case (1) for the sterilization and conservation of thermolabile endoscopes according to claim 1, wherein said reversible fastening means comprise a strip (14) of inextensible material provided with tightening means (15), adapted to tighten the edges of said first (10) and said second (20) shell to keep them stably joined to one another.

14. The case (1) for the sterilization and conservation of thermolabile endoscopes according to claim 13, wherein said strip (14) comprises said handle (5) and said supporting feet.

15. The case (1) for the sterilization and conservation of thermolabile endoscopes according to claim 13, wherein said strip (14) comprises means for form coupling with said machine.

16. The case (1) for the sterilization and conservation of thermolabile endoscopes according to claim 1, further comprising a system for the identification and/or automatic storage of information relating to said case and/or said endoscopes.

17. The case (1) for the sterilization and conservation of thermolabile endoscopes according to claim 7, wherein said composite material also comprises fibers chosen from fibers of Kevlar, glass, or metallic wires.
